(19) United States
(12) Patent Application Publication
(10) Pub. No.: US 2023/0000807 A1
(43) Pub. Date: Jan. 5, 2023

(54) CBD ORAL FORMULATION

(71) Applicant: Clear Lake Research, LLC, Palo Alto, CA (US)

(72) Inventor: Douglas Thomas, Palo Alto, CA (US)

(21) Appl. No.: 17/777,687

(22) PCT Filed: Jan. 19, 2021
(86) PCT No.: PCT/US2021/014020

§ 371 Date: May 18, 2022

Related U.S. Application Data
(60) Provisional application No. 62/962,925, filed on Jan. 17, 2020.

(51) Int. Cl. A61K 31/198; A61K 31/05; A61K 31/522; A61K 47/10; A61K 47/08; A61K 31/20; A61K 31/4415; A61K 47/02; A61K 31/352; A61K 31/245; A61K 31/167; A61K 31/357

(52) U.S. Cl. CPC A61K 31/198; A61K 31/05; A61K 31/522; A61K 47/10; A61K 47/08; A61K 31/20; A61K 31/4415; A61K 47/02; A61K 31/352; A61K 31/245; A61K 31/167; A61K 31/357

(57) ABSTRACT

A product comprising theanine, caffeine, and CBD is disclosed. Alternatives to the specific components are also described. A product comprising theanine, pyridoxine triacetate, and CBD is also disclosed. Alternatives to the specific components are also described. In an embodiment, an oral thin film strip delivery system includes a polymer, muco-adhesive, or other component that allows the product to dissolve in the mouth.

### BACKGROUND

Two primary endocannabinoid receptors have been identified: CB₁, first cloned in 1990; and CB₂, cloned in 1993. CB₁ receptors are found predominantly in the brain and nervous system, as well as in peripheral organs and tissues, and are the main molecular target of the endocannabinoid ligand (binding molecule), anandamide, as well as its mimetic phytocannabinoid, tetrahydrocannabinol (THC). One other main endocannabinoid is 2-arachidonoylglycerol (2-AG) which is active at both cannabinoid receptors, along with its own mimetic phytocannabinoid, cannabidiol (CBD). 2-AG and CBD are involved in the regulation of appetite, immune system functions, and pain management. “Cannabidiol: An Overview of Some Pharmacological Aspects,” by Mechoulam et al. (Journal of Clinical Pharmacology, Vol. 42, No. S1, pp. 11S-19S, November 2002) is hereby incorporated by reference herein.

### DETAILED DESCRIPTION

Oral formulations include oil drops, tinctures, thin films, and gum, to name several. The oral formulations include anti-anxiety/mood improvement, concentration/study aid, and sleep aid formulations. An oral thin film strip delivery system can include a polymer (e.g., gelatin strip, pectin strip, or carrageenan strip, to name several), muco-adhesive, or some other component that allows the product to dissolve in the mouth. In an alternative, oral thin films do not dissolve in the mouth. Where a distinction is desired, those that dissolve in the mouth can be referred to as “mouth dissolving films.”

In a specific implementation, a tincture has 40% to 60% alcohol as a suspension with polar and nonpolar aspects. In an alternative, the range is 10% to 80% for more concentrated/dilute bioactives. Although perhaps technically inaccurate, some people may refer to an “oil tincture” as a product that is between a tincture and an oil drop. In a specific implementation, an oil drop includes a hemp seed emulsion. In an alternative, the oil drop includes another oil, such jojoba, avocado, or olive oil, to name several. Short fatty acids can act as penetration enhancers (e.g., eucalyptol, limonene). In an alternative, bioactives are suspended in toilet water.

In a specific implementation, instead of a polymer, a food-grade emulsifier is used in soft gels (see the GRAS list) to create a product for consumption. A carrier oil can be used to dissolve gel-soluble components and water to dissolve water-soluble components. A filler can be used to bring into a 600 mg space to deliver an “oval 4” soft gel capsule, or some other standardized, larger or smaller, gel.

A gum, such as guar mastic, gum Arabic, wax, chicle, to name several, can be excellent for delivering potassium nitrate or some other component used to address pain, including sensitive teeth, in the mouth, and is also excellent for slow release of components to address inflammation, bad breath, decay, or coloration of teeth. Garlic, hone, ginger, clove, and oregano are all unregulated and useful ingredients. Bacteria can be addressed with an antibacterial, such as metronidazole, penicillin, or amoxicillin, to name several. Coloration of teeth can be addressed with an ingredient known to whiten teeth, such as abrasive particles, carbamide peroxide (which breaks down into hydrogen peroxide and urea), or hydrogen peroxide, to name several.

An example of a product (concentration, wake-up) will now be discussed. The specific implementation described can be characterized as a thin film strip formulation with:
100 mg CBD
100 mg caffeine
200 mg theanine

CBD has dual-activity in that it addresses both pain and inflammation. “Cannabidiol: From an Inactive Cannabinoid to a Drug with Wide Spectrum of Action,” by Zuardi (Brazilian Journal of Psychiatry, Vo. 30, No. 3, pp. 271-280, September 2008); “An Entourage Effect: Inactive Endogenous Fatty Acid Glycerol Esters Enhance 2-Arachidonoyl-Glycerol Cannabinoid Activity,” by Ben-Shabat et al. (European Journal of Pharmacology, Vol. 353, No. 1, pp. 23-31, July 1998); and “2-Arachidonoylglycerol (2-AG) Membrane Transport: History and Outlook,” by Hermann et al. (The AAPS Journal, Vol. 8, No. 2, pp. E409-E412, June 2006) are hereby incorporated by reference herein. Among other features, CBD binds to the CB₂ receptor on T-Cells, causing a reduction in inflammatory mediators, and binds to the transient receptor potential cation channel subfamily V member 1 (TrpV1), which is a pain receptor also known as the capsaicin receptor and the vanilloid receptor 1. Thus, CBD can be characterized as operating on the cell level to reduce both pain and inflammation.

Because CB₂ receptors have a low affinity for CBD, a relatively large amount of CBD should be used for efficacy. In a specific implementation, a thin film strip has 100 mg CBD. In alternatives, the oral thin film is as little as 20 mg CBD. Because CBD is a relatively expensive component, the ratio of CBD to other components is more prone to cost/efficacy trade-offs than other components of the formulation. Advantageously, a low does of CBD, of 50 mg or less, acts as a stimulant, making a 20 mg to 50 mg formulation appropriate for formulations that have a goal of reducing the amount of CBD necessary and meeting one of the goals of a concentration improver/mood enhancer, which is stimulation.

A moderate dose of CBD, of 50 mg to 100 mg, can act as a sedative. While this may seem to act in cross-purposes to the stimulant effect desired in a product designed to, at least, improve wakefulness, it has been found the sedative effect reduces jitters, which are a side effect of stimulants like caffeine, particularly with overindulgence. Accordingly, the example provided here includes a 100 mg does of CBD.

A greater proportion of CBD may be desirable for the purpose of binding to theorized CBD3 receptors or other presently poorly defined receptors with less affinity for CBD. A high dose of CBD, over 100 mg up to less than 250 mg can have a sedative effect that may be effective for certain individuals, though it is unlikely desirable to reach 250 mg. If CBD is replaced with by endocannabinoid with (an estimated) 1,000 times the affinity of CBD, such as 2-Lineoleoyl-glycerol (3443-82-1) or 2-Palmitolyl-glycerol (23470-00-0), an alternative implementation of the oral thin film could have 0.025 mg to 0.1 mg endocannabinoid, but the formulation would likely be much more expensive than CBD.

CBD has a log P of 7, which means CBD is extremely penetrating. (The partition coefficient, P, is defined as a particular ratio of the concentrations of a solute between the two solvents, a biphase of liquid phases, specifically for un-ionized solutes, and the logarithm of the ratio is thus log P.) Other endocannabinoids also typically have relatively high log P. It typically takes about 30 minutes for CBD, applied topically, to have a significant pain relief impact (though the onset of pain relief may be somewhat faster when applied to mucosa than it is when applied to skin and some endocannabinoids may have slightly different log P, which can impact the time).

For a more immediate impact, the CBD can be combined with menthol, which also binds to the TrpV1 receptor, but with an immediate response. Less than 1% menthol is necessary to avoid a product from acquiring an OTC designation; 1% or higher concentration of menthol is allowed in OTC medicines. Camphor can be used in lieu of (or in addition to) menthol but is not as effective and can cause a rash. Some products include both menthol and camphor to increase effectiveness without exceeding limits imposed on the components (e.g., to fall within OTC limits for both of the components). In a specific implementation, an oral thin film is nearly 1% menthol (just below the limit that would cause the formulation to be governed by pharmaceutical laws and regulations), but anything over about 0.1% menthol would likely have some effect. Of course, if the formulation is for an OTC product, up to the regulated limits of menthol can be used, if desired.

An oral thin film can include an endocannabinoid mimetic, such as a synthetic cannabinoid, as a component. Examples of endocannabinoid mimetics are 18-Hydroxypalmitic acid (Juniperic acid), AM404 (18718-77-6), and guineesine. In an alternative that includes, e.g., Juniperic acid and CBD, the Juniperic acid enables the CBD to linger for longer. Advantageously, because the “side effects” are reduced pain and inflammation, the composition would be safe as a topical treatment.

Alternatives to caffeine include theophylline, theobromine, or guarana, to name several. 100 mg of caffeine is believed to be a good dosage for an oral thin film product, such as described in this example, because that is about the dosage of a typical serving of coffee (at least nowadays). In an alternative, a 5 mg “decaf” option could be provided, which may have a pharmacological effect for those who don't regularly consume caffeine. In another alternative, a 35 mg “weak coffee” option could be used which would have a pharmacological effect for most consumers of the product. In another alternative, a 250 mg “instant energy” option could provide maximum pharmacological effect without a high risk of introducing jitters.

As a point of comparison, coffee is dissolved through the digestive tract of a human who consumes it and generally takes 25 minutes to have an impact. Coffee is emulsified in the small intestines, which results in 45 minutes for 99% bioeffect. A similar result could be expected for a soft gel. Advantageously, the oral thin film strip can deliver caffeine through the bloodstream for much more rapid results. Caffeine suspended in the oral thin film can be substantially completely dissolved in 30 seconds to 2 minutes (99%) after the oral thin film is put in the mouth.

The name “theanine” without a prefix generally implies the enantiomer L-theanine, which is the form found in tea leaves and as a dietary supplement ingredient. It has been found that the combination of caffeine and theanine can improve word recognition, attention switching, mood, distractibility, relative to baseline. In a specific implementation, the ratio of caffeine to theanine is 1:2, which is believed to provide excellent correlation between dosage and the just-mentioned improvements. It is not yet clear whether combination of caffeine alternatives, such as described above, with theanine would be effective, but signs point to effectiveness.

Emulsifiers typically have a polar (hydrophilic) and a non-polar (lipophilic) part and are used to increase kinetic stability. Emulsifiers that are more soluble in water are generally used to form O/W solutions and emulsifiers that are more soluble in oil are generally used to form W/O emulsions. An oil-in-water (O/W) solution is creamy and is generally better for hydration than a water-in-oil (W/O) solution (and may be considered to have a more desirable touch and feel). Because these properties are relatively unimportant for an oral thin film formulation, in a specific implementation, an oral thin film does not have an emulsifier. That said, emulsifiers allow mixture of oil and water useful for consistency in manufacturing (and for aesthetics), so an alternative includes 1 mg to 4 mg of an emulsifier.

A sleep aid will now be discussed. The specific implementation described can be characterized as an oral thin film formulation with:
100 mg CBD
2 mg pyridoxine triacetate
100 mg theanine

The advantages of CBD are similar to those described above in the first example. Notably, a moderate does is used for each, though in this example, the sedative effect is not counter balanced with a stimulant.

Pyridoxine triacetate dosage can be reduced from the example provided here, but should exceed 0.1 mg to have pharmacological effect. Advantageously, B6 helps the body product neurotransmitters (e.g., serotonin) without requiring consumption of an anti-anxiety drug; it can block glutamate and help the brain learn to calm. As a point of comparison, a person who consumes B6 can derive 75% of the bioactive in the stomach, while an oral thin film delivery system yields 99%.

As high as 50 mg pyridoxine triacetate could continue to see mood elevation effects, though beyond 50 mg the therapeutic advantage is not expected to increase. Alcoholism, liver disease, and some drugs can cause low levels of B6, making a higher does for certain patients useful. It should also be noted that 100 mg is the maximum daily allowance for B6 (30 mg for a 1 year old), making a dosage below 30 mg desirable, with 3 mg being a safe option for an OTC 10-strip package because, even if consumed by a child, it would be safe.

Advantageously, pyridoxine triacetate is more effective at going across mucosal barriers than other forms of vitamin B6 and, because the described oral thin film product is expected to capture 99%, an extremely accurate dose of B6 can be administered. This can be useful for addressing women's health concerns. For example, it is desirable to control the intake of certain substances, such as folate, B6, and B12, during pregnancy. In alternative implementation of the product provided in this example, issues associated with post-menopausal syndrome (PMS) can be addressed by adding magnesium in an approximately 4:1 ratio magnesium to pyridoxine triacetate.

Theanine, as noted above, improves cognition synergistically with caffeine. In this example, it is undesirable to stimulate the consumer of the product. However, theanine can still be used as a mood enhancer. The desired ratio of pyridoxine triacetate to theanine is about 1:50.

Alternatives to theanine include THC (which has the added benefit of “entourage effect” from a medicinal marijuana product, if that is desired), procaine, or the like.

1. A formulation comprising:
10 mg to 500 mg theanine per dose;
20 mg to 250 mg mimetic phytocannabinoid per dose;
a stimulant.

2. The formulation of claim 1 wherein the mimetic phytocannabinoid includes 20 mg to 50 mg cannabidiol.

3. The formulation of claim 1 wherein the mimetic phytocannabinoid includes 50 mg to 100 mg cannabidiol.

4. The formulation of claim 1 wherein the mimetic phytocannabinoid includes 100 mg to 250 mg cannabidiol.

5. The formulation of claim 1 comprising 10 mg to 70 mg theanine per dose, wherein the stimulant includes 5 mg to 35 mg caffeine.

6. The formulation of claim 1 comprising 70 mg to 200 mg theanine per dose, wherein the stimulant includes 35 mg to 100 mg caffeine.

7. The formulation of claim 1 comprising 200 mg to 500 mg theanine, wherein the stimulant includes 100 mg to 250 mg caffeine.

8. The formulation of claim 1 wherein the stimulant includes a stimulant selected from the group consisting of caffeine, theophylline, theobromine, guarana, and a combination of these.

9. The formulation of claim 1 comprising less than 1% menthol per dose.

10. The formulation of claim 1 comprising 1% menthol or more per dose.

11. The formulation of claim 1 comprising camphor.

12. The formulation of claim 1 comprising 1 mg to 4 mg of an emulsifier with a polar part and a nonpolar part.

13. The formulation of claim 1 comprising a penetration enhancer.

14. The formulation of claim 1 comprising a component to address inflammation, bad breath, decay, or coloration of teeth, the component to address inflammation, bad breath, decay, or coloration of teeth selected from a group consisting of garlic, hone, ginger, clove, oregano, an antibacterial, abrasive particles, carbamide peroxide, hydrogen peroxide, and a combination of these.

15. The formulation of claim 1 comprising an endocannabinoid mimetic selected from a group consisting of 18-Hydroxypalmitic acid, AM404 (18718-77-6), and guineesine.

16. A formulation comprising:
0.4 mg to 50 mg pyridoxine triacetate per dose;
20 mg to 250 mg theanine per dose;
20 mg to 250 mg mimetic phytocannabinoid per dose.

17. The formulation of claim 16 comprising 0.4 mg to 3 mg pyridoxine triacetate and 20 mg to 150 mg theanine per dose.

18. The formulation of claim 16 comprising 2 mg to 30 mg pyridoxine triacetate and 100 mg to 1,500 mg theanine per dose.

19. The formulation of claim 16 comprising 80 mg to 1,000 mg magnesium per dose.

20. A formulation comprising:
0.4 mg to 50 mg pyridoxine triacetate per dose;
a mood enhancer selected from a group consisting of theanine, tetrahydrocannabinol, or procaine;
20 mg to 250 mg mimetic phytocannabinoid per dose.
